(19) United States
(12) Patent Application Publication
(10) Pub. No.: US 2011/0183370 A1
(43) Pub. Date: Jul. 28, 2011

(54) OPTICAL IMAGING FOR IDENTIFYING CELLS LABELED WITH FLUORESCENT NANOPARTICLES

(75) Inventors: Isabelle NOISEUX, St-Augustin-de-Desmaures (CA); Pascal GALLANT, Quebec (CA); Ozzy MERMUT, Quebec (CA); Marcia VERNON, Hamilton (CA)

(73) Assignee: INSTITUT NATIONAL D'OPTIQUE, Ste-Foy (CA)

(21) Appl. No.: 13/011,239

(22) Filed: Jan. 21, 2011

Related U.S. Application Data
(60) Provisional application No. 61/297,328, filed on Jan. 22, 2010.

(51) Int. Cl. C12Q 1/04; C12M 1/34

(52) U.S. Cl. 435/34; 435/288.7

(57) ABSTRACT

An optical detection system for identifying target bacterial cells in a food sample, comprising a first fluorescent nanoparticle dye labeling specifically the target cells. A second fluorescent dye reacts non-specifically with all bacterial cells. An imaging unit optically excites the fluorescent dyes and captures images of the sample for each of two different wavelength bands emitted as a result of the excitation of the dyes. A processing unit identifies the target cells from the sample and comprises an image processing unit for processing the images of the sample. A target cell identifier identifies the target cells in the images by detecting individual units present at a same location in both of the processed images. An output unit provides data related to the identified target cells in the sample. A method is also provided.

### FIELD OF THE APPLICATION

The present application relates to a system and method for imaging and quantifying target cells, such as bacterial cells, using fluorescent dyes, and associated algorithms to selectively count targeted cells.

### BACKGROUND OF THE ART

It is known to apply fluorescent dyes to food samples so as to amplify fluorescent signals, to identify target cells in samples. In such cases, fluorescent nanoparticles (FNP) are bound to bacterial cells, thereby enabling the bacterial cells to be detected when excited with light of suitable wavelength. However, it is found that residual unbound free FNPs may not be completely removed by classical physical separation methods commonly used in bio-assays, and that existing readers may not clearly discriminate signals of FNP labeled cells from signals of unbound FNPs. Each FNP contains thousands of dye molecules allowing amplification of the fluorescence signal. However, these factors result in high background counts considering the unbound FNPs, particularly when target cell concentrations are lower than 10⁴ CFU/mL. The difficulty resides in discriminating counts of target cells from counts of residual unbound FNPs.

Standard techniques of biological amplification (e.g. using PCR amplification) are required to increase the population of the species of interest in the sample, such that a detectable optical signal can be detected. These amplification techniques require many hours (up to 48) for an acceptable population to be reached. Using the highly-fluorescing nanoparticles can reduce this population requirement and speed up the sample enrichment process. However, most inspection devices are currently based on fluorescence intensity measurements to establish the amount of the target species in the sample.

For instance, it may be desired to provide a system for detecting the level of pathogens, for example Listeria or any other pathogen, in a food matrix, to determine if the food product meets regulatory limits that are typically set at <1 per 25 g of food. It is desired to provide a system that performs such function with minimal enrichment requirements while providing enhanced sensitivity.

### SUMMARY OF THE APPLICATION

It is therefore an aim of the present disclosure to provide a novel system and a novel method for imaging and quantifying target cells, such as bacterial cells, using labeling with fluorescent nanoparticles.

Therefore, in accordance with the present application, there is provided an optical detection system for identifying target bacterial cells in a food sample, comprising: a first fluorescent nanoparticle dye to mix to the sample, the first fluorescent nanoparticle dye labeling specifically the target cells; a second fluorescent dye to add to the sample, the second dye reacting non-specifically with all bacterial cells; an imaging unit for optically exciting the fluorescent dyes and for capturing at least one image of the sample for each of two different wavelength bands emitted as a result of the excitation of the dyes; a processing unit for identifying the target cells from the sample, the processing unit comprising: an image processing unit for processing the images of the sample; and a target cell identifier for identifying the target cells in the images by detecting individual units present at a same location in both of the processed images; an output unit for providing data related to the identified target cells in the sample.

Further in accordance with the present application, there is provided a method for identifying target bacterial cells in a food sample, said method comprising: receiving a sample with target bacterial cells, the sample being mixed with a first fluorescent nanoparticle dye labeling specifically the target cells, and mixed with a second fluorescent dye reacting with all bacterial cells; illuminating the sample to optically excite the first fluorescent nanoparticle dye and the second fluorescent dye; capturing at least one image of the sample at a first wavelength band resulting from the excitation of the first fluorescent nanoparticle dye; capturing at least one image of the sample at a second wavelength band resulting from the excitation of the second fluorescent dye; identifying the target cells in the images by detecting individual units present at a same location in said images of both said wavelength bands; and outputting data related to the identified target cells in the sample.

### BRIEF DESCRIPTION OF THE DRAWINGS

FIG. 1 is a block diagram of an optical detection system for bacterial cells in accordance with an embodiment of the present application;

FIG. 2 is a method for identifying bacterial target cells using dual tagging with fluorescent dyes, in accordance with another embodiment of the present application; and

FIGS. 3A to 3D are schematic sequential views of dual tagging using the system and/or method of FIGS. 1 and 2.

### DESCRIPTION OF THE PREFERRED EMBODIMENTS

The methodology described herein is used to quantitatively recover and count targeted cells in pure culture, in mixtures of non-target bacteria and in spiked pre-enriched food samples, including milk, meat, environmental swab and vegetable, among possibilities. The procedure is simple and can be completed efficiently.

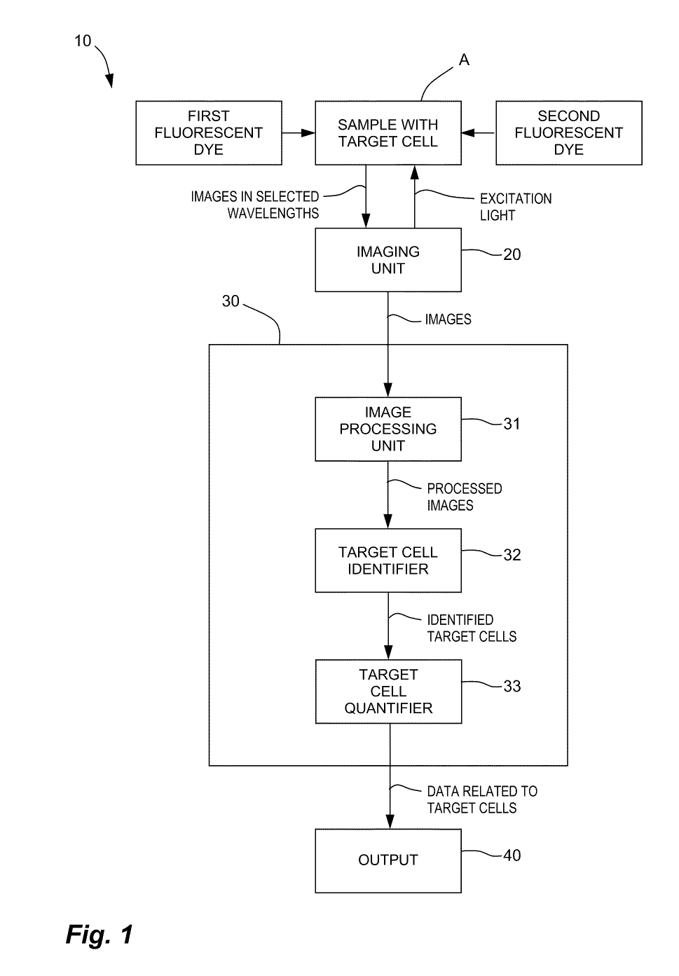

Referring to the drawings and more particularly to FIG. 1, an optical detection system for targeted bacterial cells is generally shown at 10. The optical detection system 10 is used to identify target cells in a sample A. The sample A contains the target cells and is typically supported by a slide or substrate.

According to one embodiment, the optical detection system 10 comprises an imaging unit 20, an optical detection processor unit 30, and an output unit 40.

The imaging unit 20 obtains images of the sample A with fluorescent dyes in the sample A optically excited at different wavelengths by illumination from a light source associated with the imaging unit 20.

The optical detection processing unit 30 identifies target cells from the images of the imaging unit 20.

The output unit 40 produces quantitative data pertaining to the possible presence of the target cells in the sample.

Referring to FIG. 1, two different fluorescent dyes are added to the sample (hereinafter, referred to as dual tagging). The antibodies to be conjugated with the FNPs are selected as a function of the bacterial target cells being analyzed.

Target cells are preferably bacteria comprised in a sample such as food, milk, meat, environmental swab and/or vegetables. Some bacteria can cause food poisoning, either directly or by the toxin they produce. Some of the most common food pathogens include Salmonella, Listeria monocytogenes, E. coli, Shigella, Staphylococcus, and Clostridium perfringens. Many bacterial sources of food poisoning can be found in undercooked foods such as meats, poultry, fish, and other foods such as eggs, dairy, processed meats, custards, cream pies, contaminated water, and the like. Thus, the antibodies to be conjugated with the FNPs are selected as a function of the target bacterial cells being analyzed, which are preferably bacteria such as, but not limited to, Listeria monocytogenes, Salmonella, Clostridium perfringens, E. coli, Staphylococcus aureus, Campylobacter jejuni and/or Toxoplasma gondii. Any other bacteria may also be detected by the method and system of the present disclosure.

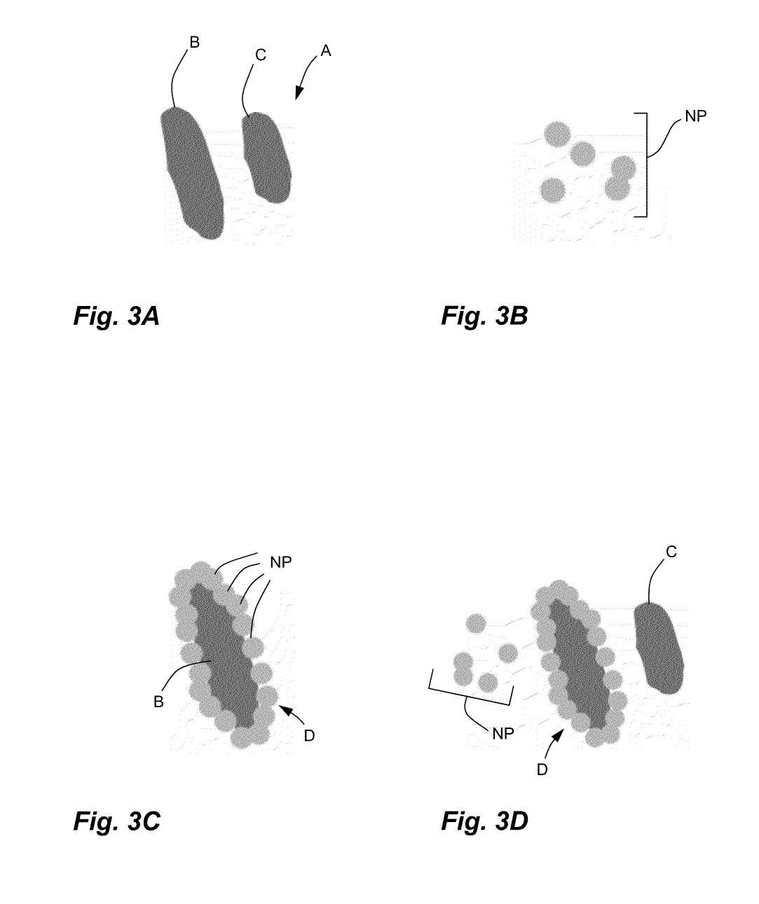

According to one non-limitative embodiment, with reference to FIGS. 3A to 3D, the system 10 is used to detect the target bacterial cell Listeria monocytogenes identified by B, in presence of other bacteria C such as Salmonella and E. coli in the sample A. A first dye, namely a nanoparticle dye features FNPs that will couple with antibodies to allow specific recognition of Listeria monocytogenes targets, as identified by D. FNPs are typically particle dyes having a size in the order of 100 nm or less. The bioconjugated FNPs contain thousands of first fluorescent dye molecules that emit fluorescence in a first wavelength band when excited with light of suitable wavelength. The binding of FNPs to individual bacterial cells enables significant amplification of the fluorescent signal because each FNP contains thousands of fluorescent dye particles (see FIG. 3B). It may be appropriate to facilitate the separation of the target cells from non-target organisms and food particles, for instance by centrifugation, filtration or the like, to reduce the amount of non-target organisms.

A second fluorescent dye for the detection of Listeria monocytogenes as set forth above, such as Syto dye, may be used (see FIG. 3A). The suitable second fluorescent dye becomes fluorescent only when bound to DNA within a bacterial cell and excited with excitation light in the suitable wavelength, whereby both target cells B and non-target cells C emit a given wavelength signal when excited in the proper excitation fluorescence wavelength.

Although Syto dye is described above, any other suitable dye (excitable in other wavelengths) may be used as well. For example, a QD-based dye (i.e., Quantum-dot) may be used, and any color wavelength may be used.

Both Syto and FNP dyes exhibit fluorescence when excited in given optical wavelengths (e.g., 488-nm blue wavelength and 532-nm green wavelength respectively in the present example). An optimal choice of fluorescent dyes allows the emission of each one in two different and non-overlapping wavelength bands.

Referring to FIG. 1, the imaging unit 20 obtains high-resolution images of the sample A. When reference is made to high resolution for the imaging unit 20, it is intended to refer to an imaging resolution that is sufficient to optically resolve each unit of target specimen or an agent targeted by the assay. In the present embodiment, the imaging unit 20 allows the imaging of bacterial cells, and therefore individuals of micron size.

The sample may often be too large to be imaged with a single capture, whereby a plurality of images of the sample may be taken for a single excitation wavelength. Moreover, in the event that numerous samples are analyzed, for each sample, at least two pictures may be obtained sequentially. The images are in epi-fluorescence, one with excitation of the first dye (e.g., blue—488 nm) and the other one with excitation of the second dye (e.g., green—532 nm). The images are preferably captured on precisely the same area of the sample, for subsequent processing. Also, it may be desired to capture an image in phase contrast for validation purposes.

The imaging unit 20 is typically a device with a microscopy configuration, having or used in combination with a suitable illumination source (e.g., lasers, LEDs, appropriate lamps with filters, such as halogen or incandescent lamps) to excite the dyes at the selected wavelengths. In the embodiment set forth above, all images are captured using magnifications ranging between 10× and 150×, for instance, although other range limits may be used as well. The imaging unit 20 of the optical detection system 10 captures an image of the labeled target cells in two different wavelength bands corresponding to each fluorescent dye used. In an example, the imaging unit 20 may be selected for its high magnification capability that allows the collection of morphological information in addition to the fluorescence signals, with the goal of validating the assay and dual-tagging method, as described hereinafter. However, validating the assay is not required in many instances.

In the embodiment set forth above, the imaging unit obtains images of the sample at the two different excitation wavelengths: excitation at 488 nm (blue) results in emission from the Syto dye, while excitation at 532 nm (green) results in emission from the FNP dye (see FIG. 3C). At the first excitation wavelength, only FNPs are imaged, and therefore bacterial cells conjugated with the antibodies and FNPs as well as unbound FNPs are imaged. At the second excitation wavelength, all bacteria fluoresce as the DNA dye labels all bacteria without specificity. This allows the elimination of the background caused by unbound FNPs, by the identification of the items fluorescing in both wavelengths, thus identifying the target cells (see FIG. 3D). In the embodiment set forth above, the target cells identified are the Listeria monocytogenes bacteria that, according to the assay design, are the only elements labeled by both fluorescent dyes. This is schematically shown in FIG. 3.

It may be required to use proper optical filters with the imaging unit 20 to ensure that excitation in the first wavelength results in collecting fluorescence emission from the first dye only, while excitation in the second wavelength results in collecting the fluorescence emission of the second dye only. It is pointed out that the first and second dyes may be excited at the same wavelength, as long as the fluorescence emissions have different wavelength bands.

The Syto dye used in an embodiment to label all bacteria may photo-bleach quickly, so neutral-density filters may be used by the imaging unit 20 to decrease the intensity of the light shone on the sample A. This results in lower fluorescence intensity captured from the bacteria. Thus, longer acquisition time may be required to capture all labeled bacteria. Other alternatives are considered, such as reducing the intensity of illumination.

The processing unit 30 comprises a processor and runs the operation of the system 10, through appropriate user interfaces. The processing unit 30 commands the imaging unit in obtaining images for both excitation wavelengths, and processes these images to identify and quantify the presence of target cells in the sample A. The processing unit 30 runs a plurality of algorithms for the identification and/or quantification of target cells.

An image processing unit 31 receives the images from the imaging unit 20. The image processing unit 31 performs any type of processing for subsequent interpretation of the images. For instance, the images received from the imaging unit 20 at the two different wavelength channels are compared (e.g., coregistered and fused). Also, the image processing unit 31 may filter out a portion of the wavelength band of the images, for instance to avoid wavelength-band overlap in the images. Moreover, as mentioned above, in many instances the sample will be too large to be analyzed by a single image. Accordingly, various images are captured for a single excitation wavelength, and the various images are tiled by the image processing unit 31 to form a mosaic for the subsequent identification of target cells.

Still referring to FIG. 1, a target cell identifier 32 compares the processed images. The target cell identifier therefore identifies target cells from the processed images, by detecting individual items at a same location in both images, emitting fluorescence in the two separate channels. The target cell identifier 32 may therefore optically segregate the target cells from non-target cells and unbound FNPs.

The target cell quantifier 33 counts the target cells from the sample A, as identified by the target cell identifier 32. Accordingly, the cell quantifier 33 provides quantitative data related to the possible presence of target cells in the sample. The target cell quantifier 33 may run an algorithm to count such dual signal items—the target cells—in the compound image and reject those not registering this dual channel response, achieving the aforementioned optical separation digitally.

Still referring to FIG. 1, the optical detection system 10 outputs the quantitative data through the output unit 40. The output may be in any suitable form, such as cell counts, statistical data, images, pass/fail indication related to the presence or absence of pathogens, etc. With respect to a pass/fail indication, the output 40 may be programmed with a predetermined criterion that allows action/decision to be taken by the operator of the system. Alternatively, the data being output may be in the form of processed images, whereby other counting methods and apparatuses may be used to analyze the sample A.

With respect to the optical detection system 10, the combination of the excitations in both 488 and 532 nm wavelengths (and collection of light in appropriate channels), in addition to the phase contrast images and high magnification of the imaging unit 20, allows the validation of the dual-tagging discrimination concept using morphological information. This step requires additional computation time, and high magnification images.

In an embodiment, the high magnification of the imaging unit 20 is used to obtain the morphological aspect of the bacteria, to subsequently distinguish the unbound FNPs from the bacteria. The use of dual tagging and high magnification with optical excitation at a 488 nm wavelength on the imaging unit 20 allows the confirmation of the specific labeling with NP-532 dye. The present disclosure allows the optical removal of the fluorescent background caused by unbound FNPs that cannot be eliminated by the washing steps included in normal bio-assay, allowing to sensitively recover and count targeted cells, for instance in the range of 10⁴ and 10⁸ cfu/mL.

The imaging unit 20 may be designed as an epi-fluorescence microscope with enough magnification and resolution to separate individual bacteria within a sample.

It is also considered to use an imaging unit 20 having a large FOV. According to an embodiment, a high pixel count camera can potentially be used to increase image resolution to the point where magnification can be reduced without loss of optical resolving power, thus enlarging the FOV. The second embodiment favors the capture of numerous images in both fluorescence channels that cover a part of the sample. A scanning unit then moves the sample so that imaging of the different areas can be done and a tiling of the entire sample (a.k.a., mosaic) may be built prior to coregistration and counting.

These embodiments may also feature a Z-scanning system, whereas different planes of the sample can be moved in the focal plane of the device, to compensate for an inherent low depth of field of the optical configuration. In this fashion, the sample format constraints can be relaxed (i.e. thickness of the sample under study).

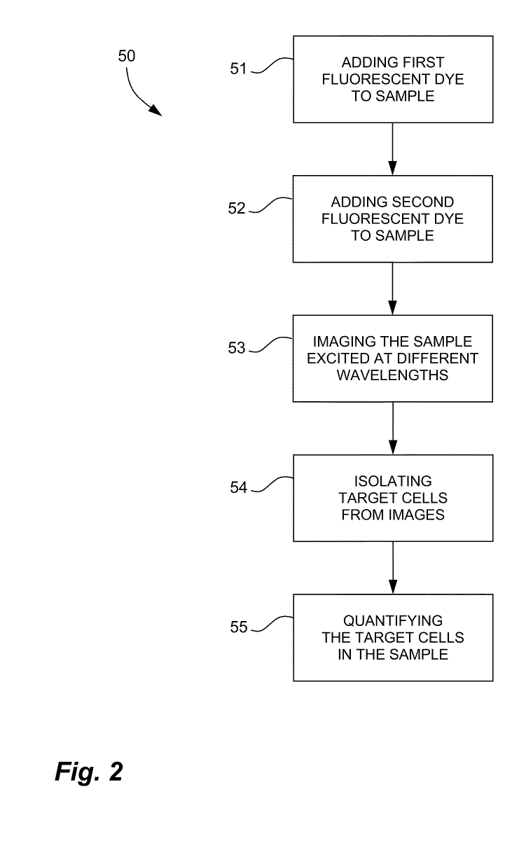

Referring to FIG. 2, a method for identifying target cells in a sample, using dual tagging, is generally shown at 50. The method may be used with the optical detection system 10.

The method 50 may be applied to a pre-treated sample. According to an embodiment, the sample to be inspected is extracted from foodstock, animal, bioculture or a patient and processed in the final inspection assay form. This step typically involves a shortened enrichment stage. The sample may be pre-treated to reach a minimal bacterial concentration for detection. For instance, in the case of food sample testing, food samples may be pre-enriched for a minimal time period to allow a single cell in 25 g (or mL) of food to multiply to a detection level. Samples that are not pre-enriched may also be used.

According to 51, a first fluorescent nanoparticle dye is added to the sample. In the embodiment set forth above, FNP dye may be used as a first fluorescent dye. In an example, a pre-enriched food broth sample is mixed with antibody-conjugated FNPs to specifically tag and capture Listeria or other pathogens. The target bacteria are specifically labeled by the FNP dye and may be treated through centrifugation and/or filtration to separate unbound FNPs and non-target cells from the target cells.

According to 52, a second fluorescent dye is added to the sample. The second fluorescent dye becomes fluorescent when bound to the DNA and excited with an appropriate wavelength to fluoresce within a different wavelength band (i.e., second wavelength band) than that caused by excitation of the first fluorescent dye (i.e., first wavelength band). In the embodiment set forth above, Syto dye may be used as second fluorescent dye.

According to 53, separate images are taken of the sample in its testing format on a sample support, with excitation in the two wavelengths. In the embodiment set forth above, the excitation in the first wavelength causes all bacteria with FNPs to be imaged, in addition to unbound FNPs, while the excitation in the second wavelength causes all bacteria to fluoresce including other bacteria than the target.

The process is automated so that the entire surface of the sample is imaged (either through high-resolution large FOV imaging or scanned/mosaic imaging as set forth above) in both fluorescence channels. In a scanned imaging embodiment, the creation of the mosaic image is performed before the processing of the images. If a z-scanning feature is implemented (as set forth above), another set of images over the whole sample surface is taken for each depth position.

According to 54, target cells are isolated, by comparing the items fluorescing at both wavelengths, from the items imaged in 53. The processed images at the two different wavelength channels are compared (e.g., coregistered and fused) to identify items in the field of view that exhibit a signal in both wavelength channels, thus identifying the target cells. In the embodiment set forth above, the target cells identified are the Listeria monocytogenes bacteria that, according to the assay design, are the only element labeled by both fluorescent dyes. According to an embodiment, the target cells are isolated by a coregistration of the images. Pattern recognition may be applied on the coregistered images to identify all fluorescing individuals. Then, an analysis of each individual recognized is made to establish if the fluorescence emitted is produced in both predetermined channels, in which case the particular individual is counted as a member of the target species.

In 55, the target cells may be quantified (i.e., counted), so as to provide quantitative data pertaining to the sample. Additionally, a validation step may be performed as well. In such a case, high magnification of the imaging unit 20 may provide enough details to perform a visual validation using morphological information and the targeted cells visually detected.

Species individuals may then be displayed and/or compared against a predetermined criterion that allows action/decision to be taken by the operator of the system, such as a Pass/Fail indication. For example, in the food inspection sector, the food stock might be identified as being contaminated by Listeria monocytogene and the production line should be stopped and inspected to avoid potential food poisoning in the population.

1. An optical detection system for identifying target bacterial cells in a food sample, comprising:
a first fluorescent nanoparticle dye to mix to the sample, the first fluorescent nanoparticle dye labeling specifically the target cells;
a second fluorescent dye to add to the sample, the second dye reacting non-specifically with all bacterial cells;
an imaging unit for optically exciting the fluorescent dyes and for capturing at least one image of the sample for each of two different wavelength bands emitted as a result of the excitation of the dyes;
a processing unit for identifying the target cells from the sample, the processing unit comprising:
an image processing unit for processing the images of the sample; and
a target cell identifier for identifying the target cells in the images by detecting individual units present at a same location in both of the processed images;
an output unit for providing data related to the identified target cells in the sample.

2. The optical detection system according to claim 1, wherein the imaging unit is a device with a microscopy configuration combined with an illumination source.

3. The optical detection system according to claim 1, further comprising a scanning unit for displacing the sample, with the imaging unit taking at least two images for each of the wavelength bands, and wherein the image processing unit processes the at least two images for each of the wavelength bands into a mosaic image.

4. The optical detection system according to claim 3, wherein the scanning unit comprises z-scanning means to focus the images at various depths below the surface of the sample, and wherein the image processing unit combines the images of the various depths for each of the wavelength bands.

5. The optical detection system according to claim 1, wherein the image processing unit performs a coregistration of the images of the sample for the two wavelength bands.

6. The optical detection system according to claim 1, further comprising optical filters in the imaging unit to capture the images within a limited wavelength band.

7. The optical detection system according to claim 1, wherein the target cell identifier validates the identification of the target cells by comparing the isolated individual units to morphological data related to target bacterial cells.

8. The optical detection system according to claim 1, wherein the processing unit further comprises a target cell quantifier for counting the identified target cells, and wherein the data of the output unit is the number of identified target cells.

9. The optical detection system according to claim 1, wherein the imaging unit is associated with an illumination source for optically exciting the first nanoparticle fluorescent dye with excitation light in a first wavelength, and the second fluorescent dye with excitation light in a second wavelength, the first and the second wavelengths being different from one another.

10. A method for identifying target bacterial cells in a food sample, said method comprising:
receiving a sample with target bacterial cells, the sample being mixed with a first fluorescent nanoparticle dye labeling specifically the target cells, and mixed with a second fluorescent dye reacting with all bacterial cells;
illuminating the sample to optically excite the first fluorescent nanoparticle dye and the second fluorescent dye;
capturing at least one image of the sample at a first wavelength band resulting from the excitation of the first fluorescent nanoparticle dye;
capturing at least one image of the sample at a second wavelength band resulting from the excitation of the second fluorescent dye;
identifying the target cells in the images by detecting individual units present at a same location in said images of both said wavelength bands; and
outputting data related to the identified target cells in the sample.

11. The method according to claim 10, wherein identifying the target cells comprises coregistering the images of the sample captured in both said wavelength bands.

12. The method according to claim 10, wherein capturing the images comprises optically filtering a fluorescent emission to capture the images in predetermined wavelength bands.

13. The method according to claim 10, further comprising validating the identification of the target cells by comparing the detected individual units to morphological data related to target bacterial cells.

14. The method according to claim 10, further comprising counting the identified target cells, and wherein outputting data comprises outputting the number of identified target cells.

15. The method according to claim 10, wherein capturing an image of the sample in both said wavelength bands comprises capturing at least two images of parts of the sample in each said wavelength band, and forming a mosaic image for each said wavelength band.

16. The method according to claim 15, wherein capturing at least two images of parts of the sample at each said wavelength band comprises capturing the at least two images at different depths.

17. The method according to claim 10, wherein illuminating the sample to optically excite the first fluorescent nanoparticle dye and the second fluorescent dye comprises illuminating the sample to optically excite the first fluorescent nanoparticle dye with excitation light in a first wavelength, and illuminating the sample to optically excite the second fluorescent dye with excitation light in a second, the first and the second wavelengths being different from one another.
